(19) United States
(12) Patent Application Publication
(10) Pub. No.: US 2026/0212770 A1
(43) Pub. Date: Jul. 23, 2026

(54) ELECTRONIC DEVICE FOR IMPLEMENTING IoT-BASED ADAPTIVE LEARNING USING EDUCATIONAL ANALYTICS STANDARDS AND METHOD OF OPERATING THE SAME

(71) Applicant: INDUSTRY ACADEMIC COOPERATION FOUNDATION, HALLYM UNIVERSITY, Chuncheon-si (KR)

(72) Inventors: Cheol Kyu SHIN, Seoul (KR); Seop Hyeong PARK, Chuncheon-si (KR); Hyun Je PARK, Seongnam-si (KR); JungMin HWANG, Seoul (KR)

(73) Assignee: INDUSTRY ACADEMIC COOPERATION FOUNDATION, HALLYM UNIVERSITY, Chuncheon-si (KR)

(21) Appl. No.: 19/033,877

(22) Filed: Jan. 22, 2025

(30) Foreign Application Priority Data
Jan. 21, 2025 (KR) 10-2025-0008758

(51) Int. Cl. G09B 5/02; A61B 5/00; A61B 5/024; A61B 5/16

(52) U.S. Cl. CPC G09B 5/02; A61B 5/0022; A61B 5/02405; A61B 5/165

(57) ABSTRACT

An electronic device for implementing IoT-based adaptive learning using educational analytics standards and a method of operating the same are disclosed. An electronic device according to the present disclosure includes at least one communication interface configured to perform a communication connection to an external device or a control target device; a memory capable of storing at least one instruction; and a processor connected to the communication interface and the memory, and the processor may acquire a real-time sensing value for a learner's state acquired through at least one sensor from an external device through the communication interface, acquire state information of the learner based on the acquired real-time sensing value and non-real-time recording data for the learner's learning activity, identify at least one educational environment and educational content corresponding to the state information, and transmit a control command for providing at least one of the identified educational environments and educational content to a target device through the communication interface.

### TECHNICAL FIELD

The present disclosure relates to technology for providing educational content using real-time sensing data for a subject, and more specifically, to an electronic device for monitoring a learner's state in real time through an IoT sensor using educational analytics standards and providing an adaptive learning environment and content.

### BACKGROUND

Project Name: 2024 Regional University Revitalization (Glocal University)—062
Period: Mar. 1, 2024~ Feb. 28, 2025

With the development of communication and network technologies, various systems that can manage personal information, user information, or the like on servers, for example, have been developed.

User terminal devices such as smartphones and tablet PCs have become widely distributed, and a plurality of user terminals may individually perform communication connections with a server to transmit various information requests to the server, and provide visual and auditory information to a user based on information received from the server.

The importance of adaptive education for providing a customized learning environment by utilizing artificial intelligence (AI) and edutech technology is being emphasized. In order to provide the adaptive education, it is important to ascertain a learner's current state in real time and adjust a level of learning difficulty.

### DISCLOSURE

### Problems to be Solved

Existing educational analytics standards such as xAPI and caliper analytics that provide adaptive education are limited to non-real-time learning activity data and thus have limitations in ascertaining real-time learning state and environment. The limitations may be overcome by utilizing IoT technology.

The present disclosure provides an electronic device that collects state information of a learner in real time based on IoT and combines the state information with non-real-time learning data to provide an adaptive learning environment and content. This implements an optimal learning environment suitable for a learner's state and maximizes an educational effect.

The objects of the present disclosure are not limited to the object mentioned above, and other objects and advantages of the present disclosure that are not mentioned can be understood from the following description and will be more clearly understood from embodiments of the present disclosure. Further, it will be easily understood that the objects and advantages of the present disclosure can be realized by the means and combinations thereof defined in the claims.

### Means to Solve the Problems

According to an aspect of the present disclosure, there is provided an electronic device for implementing IoT-based adaptive learning using educational analytics standards, the electronic device including: at least one communication interface configured to perform a communication connection to an external device or a control target device; a memory capable of storing at least one instruction; and a processor connected to the communication interface and the memory, wherein the processor acquires a real-time sensing value for a learner's state acquired through at least one sensor from an external device through the communication interface, acquires state information of the learner based on the acquired real-time sensing value and non-real-time recording data for the learner's learning activity, identifies at least one educational environment and educational content corresponding to the state information, and transmits a control command for providing at least one of the identified educational environments and educational content to a target device through the communication interface.

According to another aspect of the present disclosure, there is provided a method of operating an electronic device for implementing IoT-based adaptive learning using educational analytics standards, the method including: acquiring a real-time sensing value for a learner's state acquired through at least one sensor from an external device through the communication interface; acquiring state information of the learner based on the acquired real-time sensing value and non-real-time recording data for the learner's learning activity; identifying at least one educational environment and educational content corresponding to the state information; and transmitting a control command for providing at least one of the identified educational environments and educational content to a target device through the communication interface.

According to still another aspect of the present disclosure, there is provided a non-transitory computer-readable recording medium storing at least one instruction executed by a processor of the electronic device so that the method of controlling the electronic device.

### Advantageous Effects

According to the present disclosure, it is possible to provide a customized learning environment by comprehensively analyzing a learner's state and learning activity data. A real-time sensing value and a non-real-time sensing value are combined so that the learner's state is accurately identified and an appropriate educational environment and content are automatically adjusted, thereby maximizing learning effects.

This technology can be utilized in various educational environments such as schools, remote learning platforms, and job training, and can contribute to educational innovation by providing a personalized learning experience.

### BRIEF DESCRIPTION OF THE DRAWINGS

The aspects, features, and advantages of specific embodiments of the present disclosure will be clarified through the following description with reference to the accompanying drawings.

FIG. 1 is a block diagram illustrating a configuration of an electronic device according to an embodiment of the present disclosure.

FIG. 2 is a flowchart illustrating an operation of the electronic device according to an embodiment of the present disclosure.

FIG. 3 is a block diagram illustrating an existing data-centric learning analysis standard system according to an embodiment of the present disclosure.

FIG. 4 is a block diagram illustrating a process of acquiring a control command for educational content and an educational environment corresponding to real-time sensing values and non-real-time recording data using a data-centric learning analysis system and an adaptive learning system according to an embodiment of the present disclosure.

FIG. 5 is a block diagram illustrating a process of identifying an educational content and educational environment corresponding to biometric data using the data-centric learning analysis system and the adaptive learning system and providing the educational content and the educational environment to a learner and a teacher through a dashboard according to an embodiment of the present disclosure.

FIG. 6 is a flowchart illustrating an operation of the electronic device identifying educational content based on biometric data according to an embodiment of the present disclosure.

### DETAILED DESCRIPTION

The present embodiments may undergo various transformations and may include various embodiments, and specific embodiments will be illustrated in the drawings and described in detail in the detailed description. However, this is not intended to limit the scope of a specific embodiment, and it should be understood that various modifications, equivalents, and/or alternatives of the embodiments of the present disclosure are included. In relation to the description of the drawings, similar components may be denoted by similar reference numerals.

When a determination is made that specific description of related known functions or configurations may unnecessarily obscure the gist of the present disclosure in describing the present disclosure, detailed description thereof will be omitted.

Further, the following embodiments may be modified in various other forms, and the scope of the technical idea of the present disclosure is not limited to the following embodiments. Rather, these embodiments are provided to make the present disclosure more faithful and complete and fully convey the technical idea of the present disclosure to those skilled in the art.

The terminologies used in the present disclosure are used only to describe specific embodiments and are not intended to limit the scope of the rights. Singular expressions include plural expressions unless the context clearly indicates otherwise.

In the present disclosure, the expression “have,” “may have,” “includes,” or “may include” indicates the presence of a relevant feature (for example, a component such as a number, function, operation, or part), and do not exclude the presence of additional features.

In the present disclosure, the expression “A or B,” “at least one of A or/and B,” or “one or more of A or/and B” may include all possible combinations of items listed together. For example, “A or B,” “at least one of A and B,” or “at least one of A or B” can all refer to (1) a case in which at least one A is included, (2) a case in which at least one B is included, and (3) a case in which both at least one A and at least one B are included.

The expressions “first” and “second” as used herein can describe various components regardless of order and/or importance, and are only used to distinguish one component from others and do not limit the components.

When a component (for example, a first component) is referred to as being “operatively or communicatively coupled with/to” or “connected to” another component (for example, a second component), it should be understood that the component can be directly coupled to the other component or can be connected via another component (for example, a third component).

On the other hand, when a component (for example, a first component) is mentioned to be “directly connected” or “directly coupled” to another component (for example, a second component), it can be understood that another component (for example, a third component) is not present between the component and the other component.

The expression “set to (configured to)” used in the present disclosure may be used interchangeably with, for example, “suitable for,” “having the capacity to,” “designed to,” “adapted to,” “made to,” or “capable of” according to situations. The term “set to (configured to)” may not necessarily mean only “specifically designed to” in terms of hardware.

Instead, in some situations, the expression “a device configured to” may mean that the device “can do” together with other devices or components. For example, the phrase “a processor configured (or set) to perform A, B, and C” may mean a dedicated processor (for example, an embedded processor) for performing the operations, or a generic-purpose processor (for example, a CPU or an application processor) that can perform the operations by executing one or more software programs stored in a memory device.

In the embodiments, a “module” or “unit” may perform at least one function or operation, and may be implemented as hardware or software or as a combination of hardware and software. Further, a plurality of “modules” or a plurality of “units” may be integrated into at least one module and implemented as at least one processor, except for a “module” or “unit” that needs to be implemented as a specific hardware.

Meanwhile, various elements and areas in the drawings are schematically drawn. Therefore, the technical idea of the present disclosure is not limited by relative sizes or intervals drawn in the accompanying drawings.

Hereinafter, embodiments according to the present disclosure will be described with reference to the accompanying drawings in detail so that those skilled in the art to which the present disclosure belongs can easily implement the embodiments.

An electronic device 100 may be the electronic device 100 that may perform an operation of providing customized educational content 3 and educational environment 4 optimized for a learner by using a sensing value 1 regarding a learner's state received from various IoT devices, prestored data regarding the learner's learning, or the like.

The electronic device 100 may be, for example, a server, which is a computer that provides services to a client through a network. The server may be an FTP server, a web server, a database server, or a cloud-based server, and the server may be built with an operating system such as Linux.

The server may include a plurality of different functions, may not necessarily be a single device, and may be distributed across several devices to implement respective functions.

The electronic device 100 may also be a user terminal device. The user terminal device may include, but is not limited to, at least one of a smartphone, a tablet personal computer (PC), a laptop PC, a netbook computer, a mobile device, and a wearable device.

The electronic device 100 according to an embodiment of the present disclosure is not limited to the above-described device, and the electronic device 100 may be implemented as the electronic device 100 having two or more functions of the above-described device.

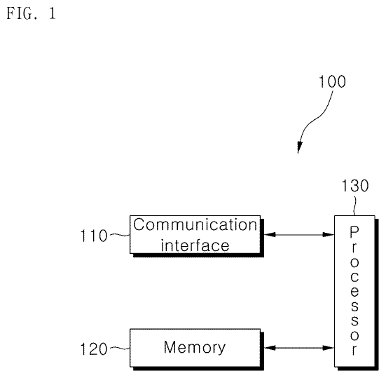

FIG. 1 is a block diagram illustrating a configuration of the electronic device 100 according to the embodiment of the present disclosure.

Referring to FIG. 1, the electronic device 100 may include a communication interface 110, a memory 120, and a processor 130. However, the configuration included in the electronic device 100 is not limited thereto, and some configurations may be omitted or other additional configurations may be added.

The communication interface 110 may include a wireless communication interface, a wired communication interface, or an input interface. The wireless communication interface may perform communication with various external devices using wireless communication technology or mobile communication technology. Such wireless communication technologies may include, for example, Bluetooth, Bluetooth Low Energy, CAN communication, Wi-Fi, Wi-Fi Direct, ultrawide band (UWB), zigbee, infrared data association (IrDA), or near field communication (NFC), and the mobile communication technology may include 3GPP, Wi-Max, Long Term Evolution (LTE), 5G, or the like.

The wireless communication interface may be implemented using an antenna, a communication chip, a substrate, or the like that may transmit electromagnetic waves to the outside or receive electromagnetic waves transmitted from the outside.

The wired communication interface may perform communication with various devices based on a wired communication network. Here, the wired communication network may be implemented using a physical cable such as a pair cable, a coaxial cable, an optical fiber cable, or an Ethernet cable, for example.

Any one of the wireless communication interface and the wired communication interface may be omitted in some embodiments. Therefore, the electronic device 100 may include only the wireless communication interface or only the wired communication interface. In addition, the electronic device 100 may include an integrated communication interface 110 that supports both a wireless connection through the wireless communication interface and a wired connection through the wired communication interface.

The electronic device 100 is not limited to including one communication interface 110 that performs one type of communication connection, and may include a plurality of communication interfaces 110 that perform a plurality of types of communication connections.

The processor 130 may receive (or acquire) a real-time sensing value 1 for the learner's state acquired through at least one sensor from external devices such as various Internet of Things (IoT) device through the communication interface 110.

In addition, the processor 130 may acquire learner's state information based on non-real-time recording data 2 of the learner's learning activities received from an external device through the communication interface 110.

The processor 130 may transmit a control command to the control target device to adjust (or provide) at least one of the educational environment 4 and the educational content 3 through the communication interface 110.

The memory 120 temporarily or non-temporarily stores various programs or data, and transmits the stored information to the processor 130 according to a call of the processor 130. Further, the memory 120 may store various types of information required for the calculation, processing, or control operations of the processor 130 in an electronic format.

The memory 120 may include, for example, at least one of a main storage device and an auxiliary storage device. The main storage device may be implemented using a semiconductor storage medium such as a ROM and/or a RAM. The ROM may include, for example, a typical ROM, EPROM, EEPROM, and/or MASK-ROM. The RAM may include, for example, a DRAM and/or SRAM. The auxiliary storage device may be implemented using at least one storage medium capable of permanently or semi-permanently storing data, such as a flash memory device, a secure digital (SD) card, a solid state drive (SSD), a hard disk drive (HDD), an optical recording medium such as a magnetic drum, a compact disc (CD), a DVD, or a laser disc, a magnetic tape, a magneto-optical disc, and/or a floppy disk.

The memory 120 may store the real-time sensing value 1 for the learner's state acquired through at least one sensor, which has been received from an external device such as various Internet of Things (IoT) devices.

The memory 120 may acquire the learner's state information based on the non-real-time recording data 2 for the learner's learning activity.

The memory 120 may store the educational environment 4 and the educational content 3 corresponding to the real-time sensing value 1 and the non-real-time recording data 2. The memory 120 may store a control command for a target device for adjusting or providing at least one of the educational environment 4 or the educational content 3.

The processor 130 controls the overall operation of the electronic device 100. Specifically, the processor 130 is connected to a configuration of the electronic device 100 including the memory 120 as described above, and may control the overall operation of the electronic device 100 by executing at least one instruction stored in the memory 120 as described above. In particular, the processor 130 can be implemented not only as one processor but also as a plurality of processors.

The processor 130 may be implemented in various ways. For example, the one or more processors 130 may include one or more of a central processing unit (CPU), a graphics processing unit (GPU), an accelerated processing unit (APU), a many integrated core (MIC), a digital signal processor (DSP), an neural processing unit (NPU), a hardware accelerator, and a machine learning accelerator. The one or more processors 130 may control one or any combination of other components of the electronic device 100, and may perform operations related to communication or data processing. The one or more processors 130 may execute one or more programs or instructions stored in the memory 120. For example, the one or more processors 130 may perform the method according to the embodiment of the present disclosure by executing one or more instructions stored in the memory 120.

When the method according to the embodiment of the present disclosure includes a plurality of operations, the plurality of operations may be performed by one processor 130 or may be performed by a plurality of processors 130. For example, when a first operation, a second operation, and a third operation are performed by the method according to an embodiment, the first operation, the second operation, and the third operation may all be performed by a first processor, or the first operation and the second operation may be performed by the first processor (for example, a general-purpose processor), and the third operation may be performed by a second processor (for example, an AI-dedicated processor).

One or more processors 130 may be implemented as a single core processor including one core, or may be implemented as one or more multi-core processors including a plurality of cores (for example, homogeneous multi-core or heterogeneous multi-core). When one or more processors 130 are implemented as a multi-core processor, each of the plurality of cores included in the multi-core processor may include a memory inside the processor, such as an on-chip memory 120, and a common cache shared by the plurality of cores may be included in the multi-core processor 130. Further, each of the plurality of cores (or some of the plurality of cores) included in the multi-core processor 130 may independently read and execute program instructions for implementing the method according to the embodiment of the present disclosure, or all or some of the plurality of cores may be linked to read and execute program instructions for implementing the method according to the embodiment of the present disclosure.

When the method according to the embodiment of the present disclosure includes a plurality of operations, the plurality of operations may be performed by one of the plurality of cores included in the multi-core processor, or may be performed by the plurality of cores. For example, when the first operation, the second operation, and the third operation are performed by the method according to the embodiment, the first operation, the second operation, and the third operation may all be performed by a first core included in the multi-core processor, or the first operation and the second operation may be performed by the first core included in the multi-core processor, and the third operation may be performed by a second core included in the multi-core processor.

In the embodiments of the present disclosure, the processor 130 may be a system on chip (SoC) in which one or more processors 130 and other electronic components are integrated, a single core processor, a multi-core processor, or a core included in the single core processor or the multi-core processor, wherein the core may be implemented as a CPU, a GPU, an APU, a MIC, a DSP, an NPU, a hardware accelerator, a machine learning accelerator, or the like, but the embodiments of the present disclosure are not limited thereto.

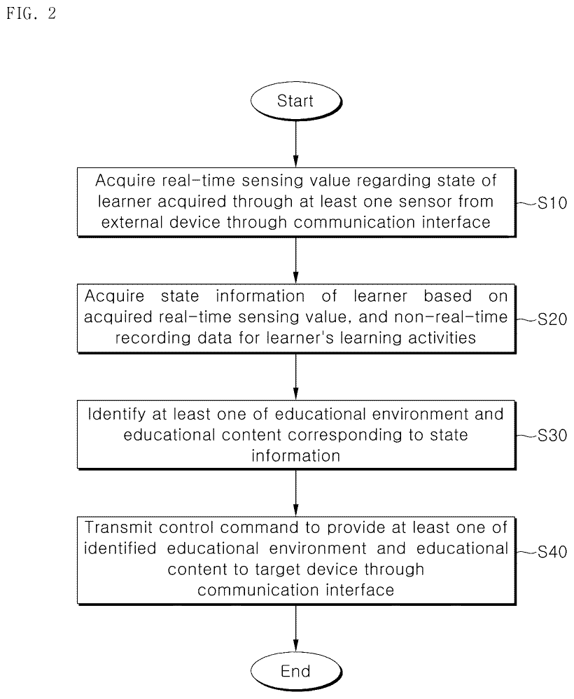

FIG. 2 is a flowchart showing an operation of the electronic device 100 according to the embodiment of the present disclosure.

Referring to FIG. 2, the processor 130 may acquire the real-time sensing value 1 regarding the state of the learner acquired through at least one sensor from an external device through the communication interface 110 (S10).

The real-time sensing value 1 means physical and physiological data measured at a current point in time through a sensor. For example, a biometric signal such as heart rate or skin temperature, or user behavioral data such as a level of screen concentration during learning corresponds to the real-time sensing value 1, but is not limited thereto.

The processor 130 may acquire state information of the learner based on the acquired real-time sensing value 1, and non-real-time recording data 2 for the learner's learning activities (S20).

The non-real-time recording data 2 is information including previous learning activity data, and may include learning time or a question solving pattern. For example, the non-real-time recording data 2 may include a learning task completion time and answer content selected during learning, but is not limited thereto.

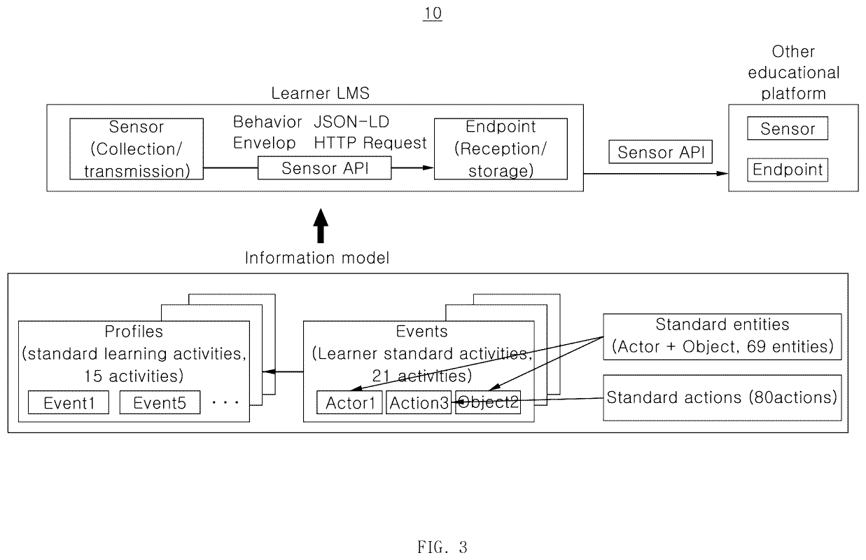

FIG. 3 is a block diagram illustrating the data-centric learning analysis system 10 according to an embodiment of the present disclosure.

Referring to FIG. 3, a structure of caliper analytics is illustrated as an example of the data-centric learning analysis system 10 (or a data-centric learning analysis standard system or a data-centric learning analysis model). The caliper analytics define learning activities through an information model, structure data, and then transmit and receive related data through a sensor application programming interface (API).

According to the existing data-centric learning analysis system 10, provided data is focused on non-real-time learning activity data such as learning activities, assignment activities, lecture activities, and assessment activities.

The information model refers to a conceptual model for structurally defining objects (entities), relationships, and properties related to a learning event and expressing these as a standardized data structure. First, in the caliper, all learning activities (for example, watching lectures, taking quizzes, writing discussion posts, or the like) are expressed as events. Referring to Table 1, 21 events are defined.

TABLE 1

| No. | Type | Description |
|---|---|---|
| 1 | Annotation | Activities of adding notes or annotations to digital |
| | | content |
| 2 | Assessment | Behavior occurring when using an assessment tool |
| | | such as online tests or quizzes (for example, solving |
| | | questions, submitting tests, grading, and receiving |
| | | grades), learner behavior throughout overall |
| | | assessment vs AssessmentItem |
| 3 | Assessment Item | Behavior when individual assessment questions are |
| | | handled (for example, reading specific questions, |
| | | answering specific questions, and moving to next |
| | | question). |
| | | Assessment vs behavior of dealing with individual |
| | | questions or problems |
| 4 | Assignable | Activity related to digital learning resources (content) |
| | | that learners are required to complete (for example, |
| | | total time spent for content utilization, the number of |
| | | accesses, average time per access) |
| 5 | Feedback | Activity of leaving informal opinions or assessments |
| | | on digital materials or to others |
| 6 | Forum | Activity of a learner participating in a forum |
| | | community (for example, subscribing, posting |
| | | messages, and commenting). A forum includes one |
| | | or more topics. When a threaded discussion is |
| | | allowed, a person can comment on other people's |
| | | messages |
| 7 | Grade | Grading or assessment activities performed by an |
| | | agent such as a person or application |
| 8 | Media | Activity related to multimedia content such as audio, |
| | | image, and video. Assignable (digital content) vs |
| | | Media (multimedia content), that is, difference in |
| | | content type and processing method |
| 9 | Message | Activity of posting a message or marking read or |
| | | unread |
| 10 | Navigation | Activity of navigating digital resources on a learning |
| | | platform (for example, moving pages or materials and |
| | | using navigation menus) |
| 11 | Questionnaire | Activity of a respondent or evaluator initiating and |
| | | submitting a survey |
| 12 | QuestionnaireItem | Activity of initiating, completing, or skipping survey |
| | | items by the respondent or evaluator |
| 13 | ResourceManagement | Activity of managing an entity (adding, correcting, |
| | | deleting, changing permissions, or the like) |
| 14 | Search | Activity of searching for information in digital |
| | | materials or software |
| 15 | Session | Activity of accessing and ending an application |
| 16 | Survey | Study method of collecting data from a group of target |
| | | respondents, including Questionnaire and |
| | | QuestionnaireItem as subordinates |
| 17 | SurveyInvitation | Activity of requesting participation in a survey |
| 18 | Thread | Activity of reading or commenting on a post within a |
| | | specific forum topic (thread) |
| 19 | ToolLaunch | Executing an external tool and recording a response |
| | | (URL or the like) after the execution |
| 20 | ToolUse | A learning tool is used, progress of learning activities |
| | | is recorded, and learning-related information such as |
| | | usage time and progress situation is included |
| 21 | View | Interaction including an objective of deeply |
| | | understanding and learning the content (note creation, |
| | | note on key content, and the like) |

Further, events also have properties as shown in Table 2 below.

TABLE 2

| Property | Description |
|---|---|
| id | A number differentiating an event |
| | UUID is expressed as a URN using a format “urn:uuid:” according |
| | to “[RFC4122]” (version 4 UUID) |
| | *UUID: A unique number given to distinguish a specific object or |
| | data from others, or a numbering rule |
| | *RFC4122: A standard rule defining how to generate and use |
| | UUIDs |
| | *Version 4 UUID: A rule for creation based on a random number, |
| | which is defined in RFC4122 |
| | *URN: A name for uniquely identifying resources |
| type | String value corresponding to a term of an event defined in an |
| | 1EdTech Caliper JSON-LD context document |
| | Set to “Event” in the case of a general event, and set by using a |
| | term (such as “NavigationEvent”) in the case of a subtype |
| | * Term: A term defined in the 1EdTech Caliper JSON-LD context |
| | document, and expanded to IRI |
| profile | A string value corresponding to a profile term value, specifying |
| | within which profile a given event should be interpreted |
| | Only one profile term value is designated in each event |
| | General event is set to GeneralProfile |
| actor | Agent who has started the event (usually a person) |
| | * Agent: A generic type representing an entity that can start or |
| | perform an action, and the agent is utilized only when there is no |
| | suitable subtype to represent an actor |
| action | An action or predicate that links an actor (subject) to an object |
| | In each event, only one action can be designated, and is limited to |
| | a set of actions defined in a relevant profile. |
| object | An action target of the actor, that is, a target that interacts with the |
| | actor |
| | * Entity: A general type, person, content, or the like that represents |
| | an object participating in learning-related activities. A subtype is |
| | defined |
| eventTime | Time when the event occurred, expressed in milliseconds as an |
| | ISO 8601 date and time value |
| | Recorded in UTC, but created in a format of YYYY-MM- |
| | DDTHH:mm:ss.SSSZ |
| edApp | Applications that generate events (for example, Zoom and |
| | Smartlead) |
| | * SoftwareApplication: Computer program, application, module, |
| | platform, and system |
| generated | Entity created as a result of an interaction (for example, when a |
| | student submits an assignment, an assignment file is generated) |
| target | Entity that represents a specific part or location within an object |
| | (for example, when a specific time period of 5 minutes and 30 |
| | seconds in an online lecture video is designated, this part is a target) |
| referrer | Referencing context, that is, an inflow path (for example, when a |
| | user clicks a link in a PDF textbook to move to a video lecture, a |
| | PDF is a referrer) |
| group | Specific group where activities take place (for example, “AI |
| | project team”) |
| | * Organization: A collection of people, which act as agents and are |
| | hierarchized into higher-level organizations |
| membership | Role and state (for example, teaching assistant) in a group |
| session | Records until a user accesses the system and starts and ends an |
| | activity |
| federatedSession | When a student moves between several systems to perform |
| | learning activities, a role is to connect the activities into a single |
| | continuous session (for example, when the student moves from an |
| | LMS to an external quiz tool, activities in both the systems are tied |
| | together and recorded through federatedSession) |
| extentions | Additional user designation properties not defined in the model |
| | Key: Defined as value |

For example, an event may be defined by properties for actor, action, and object. Specifically, an event may be defined as “Student A (actor) watched video content B (Object) (Action).” Here, the actor and the object mean standard learning entities such as people and resources related to learning activities, and are defined as 69 standard entities. Also, according to a caliper analytics standard, the action may be defined as 80 standard learning actions.

As illustrated in FIG. 3, the information model provides standardized events for each scenario as a profile unit in order to cover a wide range of learning scenarios. Referring to Table 3, a type of profile is presented.

TABLE 3

| Type | Description | Example |
|---|---|---|
| Annotation | Defined to assess learner's participation, | specific passages in eBook |
| | comprehension, and satisfaction with | are Highlighted, bookmarks |
| | digital content by analyzing annotation | are added to PDFs, notes are |
| | activities such as creating bookmarks and | made on text |
| | selecting text highlights. | |
| Assessment | Data for learner assessment pattern | Quiz score, and time taken to |
| | analysis and item creation is defined | solve questions |
| | through the number of individual question | |
| | attempts for learner assessment, | |
| | completion time, skip, response, and the | |
| | like | |
| Assignable | performance data measured based on the | Number of quiz attempts, |
| | number of attempts and time spent while | and assignment completion |
| | the learner performs the task is combined | time |
| | to define the effectiveness of the allocated | |
| | resources, learner engagement, and | |
| | comprehension | |
| Forum | Learner-to-learner discussions including | Discussion board posts are |
| | one or more threads or topics in which a | written, and comments are |
| | message can be posted and a response can | left |
| | be provided for learners participating in an | |
| | online forum community is defined. | |
| Grading | A score for assessment activity is measured | Final grade (A, B, and C) |
| | and data for comparison between learners | |
| | is defined. | |
| Media | A pattern occurring while learners | Video viewing is recorded, |
| | interacting with each other through audios, | and a specific section is |
| | images, and videos and utilizing various | watched repeatedly during |
| | media is analyzed and defined. | video playback |
| Reading | Data using e-books and text-based digital | Frequency of viewing a |
| | resources is collected, and a pattern is | lecture note, and eBook |
| | analyzed to define reading activities | reading time |
| Session | Basic metrics for platform and application | Login time and exit time |
| | usage such as learner logins, logouts, and | |
| | session times are defined. | |
| Tool Use | A concept defined to measure the tools | Frequency/time of usage of a |
| | being used, their frequency, and the | learning tool |
| | number of users as the learner utilizes the | |
| | learning tools as intended by the developer | |
| Basic | A learning activity not provided in the | Notification is confirmed, |
| | profile is defined as an event to describe an | and profile is updated |
| | interaction between an actor and an object | |
| Survey | Survey participation and response are | Satisfaction survey |
| | tracked, feedback is collected, and data for | |
| | improving learning experience is provided | |
| Feedback | A learning state is improved through | Description and additional |
| | management of feedback activities, and an | learning materials are |
| | educator can assess feedback effects | provided after quizzes |
| Resource | Resource management efficiency is | New lecture materials are |
| Management | improved through tracking of activities | additionally uploaded |
| | related to creating, correcting, and deleting | |
| | learning resources | |
| Search | What topics are of interest can be | Learning materials by topic |
| | ascertained through search activities that | are searched for and recorded |
| | the learner performs to find information | |

A sensor API for collecting event data according to a standard defined in the information model (sensor) and transmitting the event data to the endpoint is defined. Specifically, the sensor API is used to capture various activities (starting, submitting, watching, posting, or the like) that occur in a learning environment (for example, LMS, educational application, or video player) as normalized events and transmit the events to the endpoint. Here, the sensor serves to detect, generate, and transmit the events. For example, the sensor “detects” learning activities such as a learner playing a video or starting/submitting a quiz, and transmits the activities in the form of events. In this case, a plurality of events transmitted at once are grouped and defined as a behavior envelope, and actual event and entity data included and transmitted in the behavior envelope correspond to the payload.

This payload is serialized and transmitted in a JavaScript Object Notation for Linked Data (JSON-LD) format. Further, communication with the endpoint is limited to message exchange using Hypertext Transport Protocol (HTTP).

The sensor API may be used not only to transmit event data to the endpoint within a learner LMS, but also to transmit related data to other educational platforms.

The processor 130 may receive (or acquire) various types of sensing values 1 from various sensors or external devices through the communication interface 110. Such sensor values may be used to ascertain the learner's state and create a customized/adaptive learning environment. Specifically, the information presented in Table 4 may be learning-related information that can be ascertained by IoT devices.

TABLE 4

| Type | Description |
|---|---|
| Biometric signal | Heart rate, heart rate variability (HRV), electrocardiogram |
| (Biometrics) analysis | (ECG), blood pressure, skin conductivity, stress index, number |
| | of steps, sleep patterns, and the like are measured through a |
| | wearable device (smartwatch, bracelet-type sensor, and the |
| | like) |
| Emotion analysis | Whether the student is currently feeling joy, interest, distress, |
| | boredom, or the like is inferred through a facial expression |
| | recognition camera, voice tone analysis, or the like |
| Classroom environment | Classroom temperature, illuminance (brightness), air quality, |
| data | noise level, or the like is monitored in real time through IoT |
| | sensors |
| Classroom activity data | Records of use of devices such as smart board (electronic |
| | whiteboard), tablet, and laptop: Task or material reading time, |
| | and a frequency of use of a specific application are measured. |
| | Records of experiment/practice activities using an educational |
| | robot or sensor tablet: How a student manipulates the device in |
| | a practical task, and part in which the student faces difficulties |
| | are measured. |
| | Level of participation of the student such as a frequency of |
| | speech during a class, the number of presentations, and a level |
| | of participation in discussion is measured by using voice and |
| | video recognition sensors. |
| Location/movement | Ascertaining movement inside and outside a school: whether |
| information | students are participating in learning (using the library and |
| | participating in special activities) or motivation for learning is |
| | inferred through analysis of a movement route or a place of |
| | stay |
| | When and where a student is, whether the student attends or |
| | is late, or the like is automatically ascertained through a smart |
| | ID tag, an RFID chip, a beacon, or the like |
| Interaction between | When a collaborative project through smart devices is |
| students | performed, IoT sensors (microphones, location sensors, or the |
| | like) or learning platform logs are used to ascertain who has |
| | collaborated with whom and to what extent |
| Communication data | Remote classes, counseling records, or the like are |
| | automatically accumulated through an IoT device linked to a |
| | video chat platform |

When information ascertained by the IoT device is reflected in an educational analytics standard, it will be advantageous in providing customized learning. The information presented in Table 4 may be added to Table 3 above and defined as a profile of the information model, which allows real-time IoT sensor data to be extended and applied to an existing non—the real-time sensing value (1)-centered standard.

The processor 130 may update the prestored state information of the learner based on the acquired real-time sensing value 1 and non-real-time recording data 2.

An example in which the non-real-time sensing value 1 and the real-time sensing value 1 are used in xAPI or the caliper analytics and the similar data-centric learning analysis system 10 is illustrated. When the existing data-centric learning analysis system 10 operates based on non-real-time sensing value 1, the present disclosure has a difference in that the real-time sensing value 1 are additionally considered in the data-centric learning analysis system 10.

The real-time sensing value 1 may be information measured by an IoT device (terminal). Further, the information measured by the IoT device may be transmitted to the data-centric learning analysis system 10. The transmitted real-time sensing value 1 may be recorded and stored in the data-centric learning analysis system 10 in various forms.

As a first method, an item for recording the real-time sensing value 1 may be added to the event item presented in Table 1. Specifically, an event item as in Table 5 below may be added.

TABLE 5

| No. | Type | Description |
|---|---|---|
| 22 | RealtimeStatus | Real-time data provided through various |
| | | sensors and devices |

It should be noted that the names of the event items in Table 5 can be changed to other names that reflect the intent of the present disclosure.

As a second method, a method of overwriting records of the real-time data in one or more of existing event items presented in Table 1 may be used. For example, a method of additionally recording the real-time data in an existing item such as Assessment may be considered.

Further, it is possible to rapidly respond to a change in learner's current state or to provide an adaptive learning function such as adjusting a level of learning difficulty to the data-centric learning analysis system 10 in an expanded manner, by analyzing the real-time data in combination with existing non-real-time recording data 2. This makes it possible for the educator to implement a sophisticated and personalized learning experience by utilizing information collected from various IoT sensors (for example, a biometric signal, an environmental state, and a level of participation) in real time.

Next, when the real-time data is recorded in the event item, properties may be added to the event while maintaining compatibility with existing standards (xAPI, the caliper analytics, or the like).

As a first method, a new property for recording the real-time data in an event property presented in Table 2 may be defined. For example, event properties such as those in Table 6 below may be added using an extensions area of xAPI or caliper analytics or a profile extension rule.

TABLE 6

| Property | Description |
|---|---|
| Heart Rate | Student's heart rate measured by a sensor |
| Stress Index | Student's stress index measured by a sensor |
| Emotional Index | Student's emotional index measured by a sensor |
| Temperature | Temperature of a classroom measured by a sensor |
| Noise Index | Noise index of the classroom measured by a sensor |
| Speech Frequency | Student's speech frequency measured by a sensor |
| Location | Location information measured by a sensor |
| Information | |

Specifically, when the first method is used, the real-time data may be recorded in the data-centric learning analysis system 10 as “the heart rate (object) measured at time B (eventTime) of student A (actor) is C (Heart Rate).”

As a second method, a method of overwriting the event property for the real-time data with one or more items in existing event properties presented in Table 2 may be used. For example, a method of recording the measurement value of the real-time data in ‘generated’ may be considered. Specifically, when the second method is used, the real-time data may be recorded in the data-centric learning analysis system 10 as “a heart rate (object) measured at time B (eventTime) of student A (actor) is C (generated).”

In the embodiment of the present disclosure, since the real-time data measured by the IoT sensor can be integrated with the existing non-real-time recording data 2 model and stored no matter which of the two methods is applied, it is possible to immediately ascertain change in the learner's state (for example, increased heart rate and increased stress index.

This may be utilized for adaptive learning functions such as learning difficulty level adjustment, personalized learning feedback, and real-time warnings, and data collision that may occur at the time of duplicate description (for example, when several sensor values are input at the same time) may be resolved through a standard extension rule or profile management.

Further, after the real-time data is recorded as an event property in the embodiment of the present disclosure, the data-centric learning analysis system 10 may comprehensively assess the real-time data by linking the real-time data to the existing non-real-time recording data 2 through various analysis engines (for example, AI models and data mining algorithms). For example, a heart rate, stress index, assignment submission record (non-real-time), or the like can be analyzed together to assess the learning efficiency of the student, combined with classroom environment data (temperature, noise index, or the like), and utilized for real-time learning environment control (air conditioning, lighting control, or the like).

The processor 130 may identify at least one of the educational environment 4 and the educational content 3 corresponding to the state information (S30).

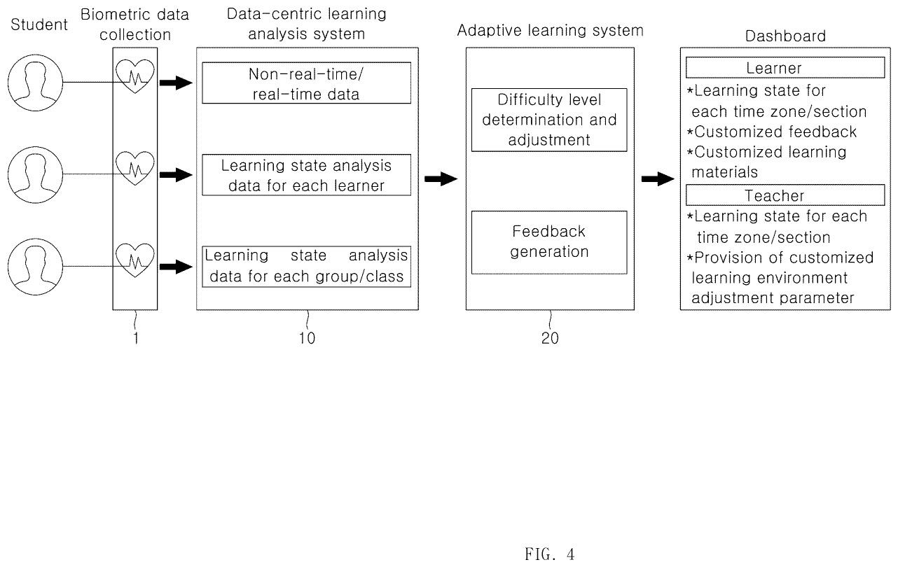

FIG. 4 is a block diagram illustrating a process of acquiring a control command for the educational content 3 and the educational environment 4 corresponding to the real-time sensing value 1 and the non-real-time recording data 2 using the data-centric learning analysis system 10 and the adaptive learning system 20 according to an embodiment of the present disclosure.

Referring to FIG. 4, the processor 130 may input the real-time sensing value 1 and the non-real-time recording data 2 to the data-centric learning analysis system 10 (or model) to acquire a database for the learner's learning activity or learner's state information.

The processor 130 may input the database of the learner's learning activities or the learner's state information to the adaptive learning system 20 (or model) to identify information for the educational content 3 and the educational environment 4.

Here, the state information may be acquired by the processor 130 from the data-centric learning analysis system 10, which is converted into a database, but is not limited thereto and may be the sensing value 1 and the non-real-time recording data 2 themselves received from an external device through the communication interface 110.

The educational environment 4 may mean a physical or digital environment in which a learning target person performs learning, and specifically, may be a temperature, humidity, illuminance, noise level, wind direction, or the like of a space in which the learner is located, but is not limited thereto.

The educational content 3 may mean all online and offline contents that the learner encounters in learning activities. For example, the educational content 3 may include educational materials according to various levels of difficulty, educational curriculum for each learning content, entertainment content, rest or meditation content, tests, quizzes, or the like.

A process of identifying information for the educational content 3 and the educational environment 4 through the adaptive learning system 20 is as follows.

In an input step, the input data may be non-real-time recording data 2 or the real-time data or learning state analysis data. The processor 130 inputs data ascertained in the input step to the adaptive learning system 20, and it is noted that this data may include all pieces of data that can be helpful for adaptive learning.

The input data is input to the adaptive learning system 20. The adaptive learning system 20 establishes a policy for customized education based on the input data. The adaptive learning system 20 may be linked with the data-centric learning analysis system 10. In other words, it is noted that the adaptive learning system 20 and the data-centric learning analysis system 10 can be combined with each other or can interact with each other to exchange data or perform specific tasks together. The policy derived from the adaptive learning system 20 may be applied to customized education in the step.

A detailed operation of the adaptive learning system 20 has been shown. The adaptive learning system 20 has a structure for performing data analysis and data processing to return a parameter for adjusting the learning environment.

Data analysis: This is a step of comprehensively interpreting the learner's state (a level of concentration, a level of understanding, a level of fatigue, or the like) and learning context (progress, learning difficulty, or the like) through machine learning/AI algorithms or statistical analysis techniques based on real-time sensor information (for example, heart rate and classroom environment temperature) and non-real-time learning records (assignment submission, assessment results, or the like).

Data processing: This is a step of generating, normalizing, and filtering learning environment adjustment parameters (for example, difficulty level adjustment value, recommended educational material list, and classroom environment setting value) based on analysis results, or reflecting these in a learning history DB or user profile through profile linkage with the data-centric learning analysis system 10, extension extensions recording, or the like.

Return of learning environment adjustment parameters: Final adjustment parameters are reflected in real time in the classroom environment (air conditioning, lighting, or the like) or learning content (level of difficulty, recommended tasks, or the like).

For example, when a learner's stress index is equal to or higher than a critical value, the level of difficulty may be lowered or additional hints may be provided, and when a classroom temperature is high, customized education may be performed by controlling an air conditioner.

This specific operation of the adaptive learning system 20 makes it possible to flexibly respond to an immediate state change (decrease in a level of concentration, increase in a level of fatigue, or the like) of each student by additionally considering the real-time sensor information, unlike an existing analysis model of ascertaining a learning state mainly based on the non-real-time recording data 2. This contributes to improving the learning efficiency and creating an educational environment 4 optimized for each student.

It should be noted that an interaction between the adaptive learning system 20 and the data-centric learning analysis system 10 used in the present embodiment may be performed through a standardized API (a sensor API, a caliper analytics/xAPI extensions, or the like) or a direct communication protocol (HTTP, WebSocket, or the like), and learning results (difficulty level classification, learning path recommendation, or the like) of an artificial intelligence model may be transmitted and shared bidirectionally. This configuration can be expanded into various forms in which the data-centric learning analysis standard and the adaptive learning system 20 are implemented simultaneously or are driven in combination.

Further, the processor 130 may identify a learner's learning state (for example, a level of concentration, a level of understanding, or a level of fatigue) and learning context (for example, progress or a level of learning difficulty) based on the acquired real-time sensing value 1 and non-real-time recording data 2.

The processor 130 may identify at least one of the educational environment 4 and the educational content 3 optimized for the learner based on the identified learning state and learning context.

According to various embodiments, the processor 130 may identify the learner's biometric data included in the acquired real-time sensing value 1. The biometric data refers to data indicating a learner's physical state, and may include, but is not limited to, a heart rate (HR), a stress index, a heart rate variability (HRV), blood pressure, skin conductance, the number of steps, a sleep pattern, brain waves, body temperature, the number of breaths per minute, or the like

The processor 130 may receive the learner's biometric data from an external device, such as a wearable device worn by the learner, at a preset period (or each time a preset event is identified).

The processor 130 may identify at least one of the level of difficulty and the composition of the educational content 3 provided to the learner based on the identified biometric data.

The level of difficulty and the composition of the educational content 3 refer to complexity and an arrangement way of learning materials, and basic concept materials for beginners or advanced question solving materials for experts correspond thereto but are not limited thereto.

The processor 130 may identify the educational content 3 corresponding to at least one of the identified level of difficulty and the composition.

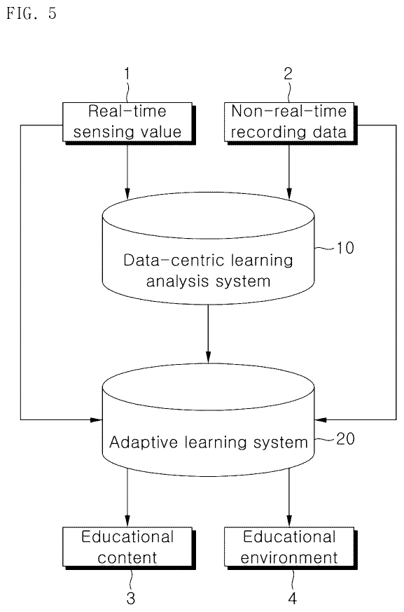

FIG. 5 is a block diagram illustrating a process of identifying the educational content 3 and the educational environment 4 corresponding to the biometric data using the data-centric learning analysis system 10 and the adaptive learning system 20 and providing the educational content 3 and the educational environment 4 to a learner and a teacher through a dashboard according to an embodiment of the present disclosure.

Referring to FIG. 5, the processor 130 may acquire biometric data information of respective students through the communication interface 110 from various IoT devices, such as user terminal devices (smart phones, or the like), wearable devices (watches, headsets, or the like), and smart devices including various sensors.

The processor 130 may receive biometric data aperiodically based on the teacher's input, or receive biometric data at a preset period.

Measurement of the biometric data may be performed at a specific point in time (question solving, quiz, lecture watching, or the like).

The processor 130 may identify various measurement indicators, scores, or the like corresponding to the biometric data. For example, in the case of an electrocardiogram, the processor 130 may acquire information on electrical activity, heart rate, rhythm, conduction time, or the like of the heart through various indicators. Further, the processor 130 may analyze concentration, stress, and a fatigue level by utilizing indicators such as a heart rate, heart rate variability, an R-R interval, and a low frequency (LF)/high frequency (HF) ratio.

The processor 130 may input the biometric data to a central learning analysis system to acquire a database for the biometric data or state information corresponding to the biometric data.

The data-centric learning analysis system 10 may analyze the learning state of the student by utilizing various types of non-real-time and real-time data.

For example, the processor 130 may record and store properties such as “heart rate”, “low frequency/high frequency ratio (LF/HF ratio)”, and “skin conductance” in the event by extending (overwriting) a structure of an extensions field of xAPI or a Real time Status event of the caliper analytics (see Table 5). This extended event information is integrated with existing non-real-time learning data (assignment submission, test score, or the like) and stored as an integrated record that allows the learner's current state to be ascertained at a glance. In other words, both the pieces of data (the non-real-time data and the real-time data) are integrated and managed by creating or overwriting an extended field with a real-time biometric signal added thereto, in addition to the area where the non-real-time activity data has been recorded in an existing table (or DB schema).

The processor 130 may acquire information obtained by analyzing the learning state of each learner based on biometric data, as well as information obtained by analyzing the learning state of each group/class.

When various types of non-real-time and real-time data are available, the processor 130 will be able to measure the learner's learning state more accurately in real time.

As illustrated in FIG. 5, the adaptive learning system 20 may be linked with the data-centric learning analysis system 10. The processor 130 may use an algorithm and artificial intelligence through the adaptive learning system 20 to determine a current level of learning difficulty and acquire a control command for adjusting the learning difficulty. Here, the control command may include an operation of “lowering/maintaining/raising” the learning difficulty. The teacher or learning system may take actions such as adjusting the difficulty or providing supplementary materials according to a corresponding scheme.

Further, the processor 130 may generate and provide appropriate feedback to the teacher or learner through the adaptive learning system 20.

The processor 130 may provide information on the educational content 3 and the educational environment 4 acquired through the data-centric learning analysis system 10 and the adaptive learning system 20 to the user through the dashboard.

In this case, the processor 130 may transmit a control command for dashboard output (or provision) to an external device, a user terminal, or the like through the communication interface 110, so that the dashboard is output on the external device and the user terminal.

The processor 130 may transmit the control command for dashboard output to each of a learner terminal and a teacher terminal.

The processor 130 may transmit a dashboard including information on the biometric data, the learner's learning state, the educational content 3, the educational environment 4, or the like to the user terminal at a preset period or whenever a preset event is identified.

However, in addition, the electronic device 100 may further include a display, and the processor 130 may provide the information on the educational content 3 and the educational environment 4 to the user in the form of a dashboard through the display.

The processor 130 may transmit customized learning feedback or learning materials based on the measured biometric data to the learner terminal.

The processor 130 may transmit a parameter for adjusting the educational environment 4 based on the biometric data measured by the teacher terminal, and various parameters may be provided as a dashboard. For example, the parameter may be a parameter related to difficulty level adjustment. For another example, the parameter may be information on providing a strategy (quiz timer, competitive element, or the like) for inducing concentration. For another example, the parameter may be a proposal for a tip (breathing exercise and simple energy consumption activity) for inducing rest.

According to various embodiments, technical characteristics are proposed in which an arousal level theory based on a Yerkes-Dodson law (inverted U-shaped curve relationship) frequently mentioned in psychology is combined with real-time biometric data and an adaptive learning algorithm, based on the fact that an appropriate level of tension (“appropriate level of stress”) can be helpful for improvement of learning performance by increasing concentration and motivation, to thereby immediately recognizing and dynamically responding to a state change experienced in a learning process by the learner, thereby improving learning performance. For example, according to the Yerkes-Dodson law, learning performance depending on the arousal level can have a relationship as shown in Table 7 below.

TABLE 7

| Arousal level | Performance |
|---|---|
| Low arousal (low tension) | Sluggishness or lack of concentration may be |
| | caused |
| High arousal (excessive | Anxiety and excessive stress interfere with |
| tension) | concentration |
| Optimal arousal (moderate | Concentration and motivation are increased |
| tension) | to maximize performance |

In the present disclosure, in order to apply the Yerkes-Dodson law to an actual learning environment, an arousal score is calculated using biometric data (heart rate (HR), stress index (SI), heart rate variability (HRV), or the like) in the step, and is divided into low arousal, high arousal, and optimal arousal depending on which section of an inverted U-shaped curve the arousal score belongs to. For example, the arousal score may be calculated as the following weighted sum.

The processor 130 may identify the learner's heart rate, stress index, and heart rate variability based on the identified biometric data.

The heart rate, stress index, and heart rate variability are indicators for measuring the learner's physical and mental states. For example, a high heart rate may indicate a learner's state of tension, or a low heart rate variability may indicate high stress, but the present disclosure is not limited thereto.

The processor 130 may identify the learner's arousal level based on the heart rate, stress index, and heart rate variability.

The arousal level is an indicator of how much the learner is concentrating, and for example, a high arousal level may indicate that intensive learning is possible, or a low arousal level may indicate that easy learning materials are needed, but the present disclosure is not limited thereto.

The processor 130 may identify a difficulty level corresponding to the arousal level. The processor 130 may identify the educational content 3 corresponding to the identified level of difficulty.

The processor 130 may calculate an arousal score corresponding to the arousal level based on Formula 1.

The arousal score is a value that numerically expresses the learner's level of concentration and learning likelihood and, for example, the arousal score may be used to determine whether to provide content with a high level of difficulty that requires concentration, or may be used as a criterion for recommending learning rest, but the present disclosure is not limited thereto.

Formula 1 is a formula for calculating the arousal score, and may include a weighted sum of heart rate, stress index, and heart rate variability.

$$Arousal score = w 1 \times heart rate + w 2 \times stress index + w 3 \times 1 / heart rate variability \qquad [ Formula 1 ]$$

w1, w2, and w3 are a first weight, a second weight, and a third weight, respectively. The heart rate may indicate the number of heartbeats during learning, the stress index may be an indicator of the learner's level of mental stress, and the heart rate variability may be an indicator indicating an amount of temporal change in the heart rates.

The first weight, the second weight, and the third weight indicate relative importance assigned to the heart rate, stress index, and heart rate variability, respectively.

The weights are adjusted to accurately reflect the characteristics of the data and the learner's state, and for example, when the heart rate has a greater influence on the level of learning concentration, a high weight may be assigned, or when the stress index is a priority analysis element, a high weight may be assigned to the index, but the present disclosure is not limited thereto.

The processor 130 may identify the learner's state as “low arousal” when the calculated arousal score is below a specific threshold range, may identify the learner's state as “high arousal” when the calculated arousal score is above the specific threshold range, and may identify the learner's state as “optimal arousal” when the calculated arousal score is in a middle range. Through such a mapping manner, it is possible to immediately ascertain the learner's arousal level by integrating biometric data measured in real time from IoT devices (wearable sensors, smartwatches, and the like) beyond simple theoretical classification.

All pieces of data that are helpful in determining the learning state and the performance (other types of data may be included in addition to the biometric data) may be utilized as input data in the input step. Therefore, the present disclosure does not limit the input data in the input step to specific data. However, biometric signal analysis and emotional analysis data presented in Table 4 may be utilized as the data input in the input step.

The arousal level of the learner may be identified based on the data input in the input step. The arousal level may be divided into X (X>0) levels. In the present disclosure, a value of X is not limited to a specific value. For example, the arousal level may be divided into three levels including low arousal, high arousal, and optimal arousal, as shown in Table 7.

Various methods for determining the arousal level based on the input data in the input step may be considered. For example, when the heart rate (HR) and the stress index (SI) are used as the input data, the processor 130 may set conditions corresponding to the low arousal, the high arousal, and the optimal arousal as follows.
Low arousal: HR<65 or SI<3
High arousal: HR>80 or SI>60
Optimal arousal: Intermediate section not corresponding to both the conditions

Further, the processor 130 may acquire information on heart rate, stress index, and heart rate variability based on deep learning to identify the learner's low arousal, high arousal, and optimal arousal.

The processor 130 may establish a learning adjustment policy in the step based on the input data. The learning adjustment policy may be used in various ways, such as adjusting the level of learning difficulty, providing supplementary learning materials, or taking a quiz. A method of establishing the learning adjustment policy in the present disclosure is not limited to a specific method. For example, the following learning adjustment policies may be reflected to three levels corresponding to low arousal, high arousal, and optimal arousal.

Low arousal: A state in which the learner is too relaxed, and a learning policy is performed in a direction of increasing the arousal level by using methods such as increasing the learning difficulty, adding fun elements, and taking quizzes.

High arousal: A state in which the learner is excessively stressed/tensioned, and the learning policy is performed in a direction of reducing the level of learning difficulty or lowering the arousal level through rest/meditation.

Optimal arousal: A state in which the learner can maintain concentration, and the learning policy is established in a direction of maximizing the learning performance by performing additional supplementary learning or describing important content.

In other words, when the arousal score calculated using first biometric data (heart rate, stress index, heart rate variability, or the like sensed during a first time period) and Formula 1 described above is less than a first threshold value, the processor 130 may identify second the educational content 3 corresponding to a higher difficulty level than that of first educational content 3 that has been previously provided. Further, in this case, the processor 130 may also identify the third educational content 3 including at least one of entertainment and quizzes.

When the arousal score calculated using the first biometric data and Formula 1 is greater than or equal to the first threshold value and smaller than the second threshold value, the processor 130 may identify fourth educational content 3 including at least one of supplementary learning and core learning. The second threshold value may be a value greater than the first threshold value.

When the arousal score calculated using the first biometric data and Formula 1 is greater than or equal to the second threshold value, the processor 130 may identify a fifth educational content 3 corresponding to a lower difficulty level than that of the first educational content 3 that has been previously provided. Further, in this case, the processor 130 may also identify sixth educational content 3 including at least one of rest and meditation.

According to the above-described embodiment, there is an effect of providing the educational content 3 with a level of difficulty and the composition suitable for the learner depending on the arousal score of the learner.

After a preset time has elapsed from a point in time when a control command for at least one of the first to sixth educational content 3 is transmitted to an external device (which may be, but is not limited to, a device that provides educational content 3 to a learner) through the communication interface 110, the processor 130 may receive second biometric data (heart rate, stress index, heart rate variability, or the like sensed during a second time period different from the first time period) through the communication interface 110.

The educational content 3 may be provided as in the above-described embodiment depending on the arousal score calculated based on the second biometric data and Formula 1.

Further, Formula 1 in which a value of the weight included in Formula 1 has been changed depending on the arousal score calculated during the preset period may be acquired, as described below. This is intended to provide the educational content 3 in which the learning efficiency of the learner can be maximized by ensuring that the arousal score is calculated in an optimized manner.

When the number of times the arousal score calculated during the preset period using the second biometric data and Formula 1 is smaller than the first threshold value or equal to or greater than the second threshold value is greater than or equal to a preset number of times, the processor 130 may acquire corrected Formula 1 including a (1-1)-th weight which is a value greater than the first weight instead of the first weight, and a (3-1)-th weight which is a value greater than the third weight instead of the third weight among the weights included in Formula 1.

That is, this is intended to increase the weight of the heart rate or heart rate variability, which is more accurate and intuitive than the stress index, so that a more accurate arousal score can be calculated.

When the number of times the arousal score calculated during the preset period using the second biometric data and Formula 1 is smaller than the first threshold value or equal to or greater than the second threshold value is smaller than the preset number of times, the processor 130 may identify Formula 1 in which there is no change in the value of each weight included in Formula 1.

In this case, the processor 130 may continuously receive (or acquire) biometric signal change measured in real time from the IoT sensor through the communication interface 110 and recalculate the arousal score or stress index through an AI model (machine learning, deep learning, or the like). The present disclosure is not limited to simply referring to the theoretical Yerkes-Dodson law, and real-time biometric signal measurement and automatic learning environment adjustment functions are implemented in an actual system. In other words, as soon as the arousal level discrimination is performed, the system may take appropriate actions such as difficulty level setting or rest proposal by itself or guide the actions to the teacher/student through the dashboard.

When a teacher input is performed, at a preset period, or each time a specific event (question solving completion, quiz end, or the like) is identified, the processor 130 may acquire the biometric data again and calculate the arousal score again, thereby adjusting learning through an iterative feedback loop.

For example, “When the learner is determined to be in a state of high arousal, rest/meditation is recommended, and when the learner is switched to “optimal arousal” as a result of performing measurement again after a few minutes, the level of difficulty is returned to a normal level.”

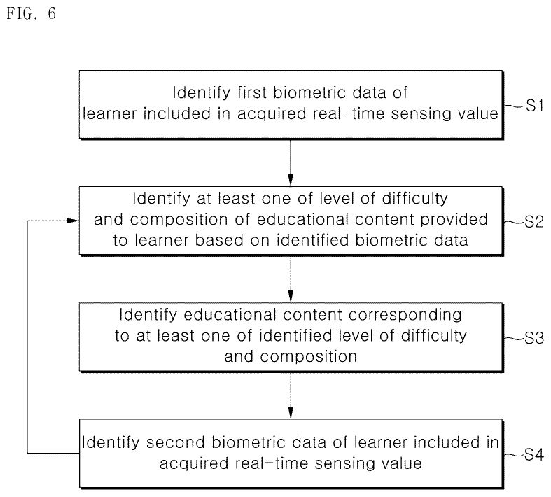

The above-described process can be summarized as a flow as in FIG. 6.

FIG. 6 is a flowchart showing an operation of the electronic device 100 identifying the educational content 3 based on biometric data according to an embodiment of the present disclosure.

Referring to FIG. 6, the processor 130 may identify the first biometric data of the learner included in the acquired real-time sensing value 1 (S1).

In this case, the processor 130 may perform a communication connection with a user terminal device or various IoT devices through the communication interface 110 to receive the biometric data of the learner.

The processor 130 may identify at least one of the level of difficulty and the composition of the educational content 3 that are provided to the learner based on the identified biometric data (S2). The processor 130 may identify the educational content 3 corresponding to at least one of the identified level of difficulty and the composition (S3).

The difficulty level of the educational content 3 may be classified according to the complexity, advancement, absolute quantity, and the like of learning content included in the educational content 3, and may be classified as high, medium, low, or classified into one or more levels.

The educational content 3 may include content for each learning step, such as concept learning, question solving, quizzes, and tests, and may include content for various fields, such as Korean, mathematics, science, English, physics, chemistry, biology, history, and economics.

The processor 130 may identify the second biometric data of the learner included in the acquired real-time sensing value 1 (S4).

The processor 130 may identify at least one of the level of difficulty and the composition of the educational content 3 provided to the learner based on the second biometric data, and identify the educational content 3 corresponding thereto.

According to various embodiments, another example of the adaptive learning algorithm utilizing the biometric data is proposed. The farther the measured biometric data is from a specific reference value, in other words, the farther the distance is, the lower the learning performance may be.

The processor 130 may identify an amount of increase in learning performance of an education target person based on Formula 2.

$$Learning performance increase amount = a - b \times distance + noise \qquad [ Formula 2 ]$$

a is the amount of increase in learning performance corresponding to a reference biometric data value of the education target person (for example, an average value of biometric data over a preset period or a reference biometric data value in a normal state), and b is a weight corresponding to a degree of learning performance decreasing according to the difference between the measured biometric data value of the education target person (for example, the sensing value 1 acquired in real time or at a preset period through a sensor, or the like) and the reference biometric data value.

The distance is a difference value between the measured biometric data value and the reference biometric data value, and noise may be a variable corresponding to randomness.

Learning performance increase amount modeling representing modeling using a principle that the learning performance decreases as the distance, that is, the biometric data moves away from the reference value, is not limited to Formula 2 described above, and it is noted that nonlinear modeling is also possible in various ways, such as giving a steep penalty as the distance increases.

The processor 130 may transmit a control command to provide at least one of the identified educational environment 4 and educational content 3 to the target device through the communication interface 110 (S40).

The target device means a device that physically implements or transforms the learning environment or content. For example, a smart lighting device or a tablet learning application corresponds to the target device, but the present disclosure is not limited thereto.

The control command may include specific operation instructions for adjusting the learning environment or content according to the identified information. For example, a command to adjust lighting brightness intensity or switch to a low-level problem corresponds thereto, and the present disclosure is not limited thereto.

The processor 130 may identify the distance as in Formula 3 below for the measurement of N different target information (biometric data).

$$distance = {\sum }_{i = 1}^{N} \vert {f}^{( i )} - {f}_{normal}^{( i )} \vert \qquad [ Formula 3 ]$$

f⁽ⁱ⁾ may be a measured biometric data value corresponding to an i-th item or target, and

$${f}_{normal}^{( i )}$$

may be a reference biometric data value corresponding to the i-th item (or target).

Here,

$${f}_{normal}^{( i )}$$

will be a value of a biometric signal that the algorithm assumes to be ideal. For example, a value of

$${f}_{normal}^{( i )}$$

may be set to an average value or median value for a biometric signal of an actual user (or the entire group). In the case of a personalized model, the value of

$${f}_{normal}^{( i )}$$

may be set to a usual biometric signal value of each user.

For example, when the heart rate (HR) and the stress index (SI) are used as input data, distance=(|HR−HRₙₒᵣₘₐₗ|+|SI−SIₙₒᵣₘₐₗ|) may be calculated according to Formula 3.

When the distance is smaller than the A value (very small deviation), the processor 130 may identify the educational content 3 corresponding to a higher level of difficulty than that of the previously provided educational content 3, or identify the educational content 3 including a reward provided to the education target person.

The distance indicates the difference between the measured biometric data value and the reference value, and may be used to analyze a change in target person's state during learning. For example, the distance may be used to measure an amount of change in heart rate or a difference in stress index, or to assess the adaptability to the learning environment, but the present disclosure is not limited thereto.

The processor 130 may identify the educational content 3 corresponding to a level of difficulty of the previously provided educational content 3 when the distance is greater than or equal to the A value and smaller than the B value (small deviation).

The A value and the B value may be reference values indicating a range of change in biometric data, and may be used to classify learner states by section and recommend appropriate learning content.

The processor 130 may identify the educational content 3 corresponding to a level of difficulty that the degree of similarity is greater than or equal to a preset value, compared to the difficulty level of the previously provided educational content 3 when the distance is greater than or equal to the B value and smaller than the C value (moderate deviation).

The level of similarity may be used as a criterion for comparison of the level of difficulty between the learning contents, and utilized to provide continuous learning experience. For example, associated problems or tasks may be recommended based on the level of difficulty and the level of similarity, or learning topics may be linked, but the present disclosure are not limited thereto.

The processor 130 may identify the educational content 3 corresponding to a lower level of difficulty than that of the previously provided educational content 3 or identify the educational content 3 including at least one of the rest and the meditation when the distance is equal to or greater than the C value (severe deviation).

The rest and the meditation may be used as content for relieving learner fatigue and reducing stress. For example, a simple deep breathing guide or short video content may be provided or linked to a meditation application, but the present disclosure is not limited thereto.

According to one embodiment, the methods according to various embodiments disclosed in the present document may be included in a computer program product and provided. The computer program product may be traded as a product between a seller and a buyer. The computer program product may be distributed in the form of a device-readable storage medium (for example, a compact disc read only memory; CD-ROM), or may be distributed online (for example, downloaded or uploaded) through an application store (for example, Play Store™) or directly between two user devices (for example, smartphones). In the case of the online distribution, at least part of the computer program product (for example, a downloadable application) may be at least temporarily stored or temporarily generated in a device-readable storage medium such as a memory of a manufacturer's server, an application store's server, or a relay server.

Although the preferred embodiments of the present disclosure have been illustrated and described above, the present disclosure is not limited to the specific embodiments described above, various modifications may be made by a person skilled in the art in the art without departing from the gist of the present disclosure claimed in the claims, and such modifications should not be individually understood from the technical idea or prospect of the present disclosure.

### DESCRIPTION OF REFERENCE NUMBERS

Although the preferred embodiments of the present disclosure have been illustrated and described above, the present disclosure is not limited to the specific embodiments described above, various modifications may be made by a person skilled in the art in the art without departing from the gist of the present disclosure claimed in the claims, and such modifications should not be individually understood from the technical idea or prospect of the present disclosure.
100: electronic device
110: communication interface
120: memory
130: processor

What is claimed is:

1. An electronic device for implementing IoT-based adaptive learning using educational analytics standards, the electronic device comprising:
at least one communication interface configured to perform a communication connection to an external device or a control target device;
a memory capable of storing at least one instruction; and
a processor connected to the communication interface and the memory, wherein
the processor
acquires a real-time sensing value for a learner's state acquired through at least one sensor from an external device through the communication interface,
acquires state information of the learner based on the acquired real-time sensing value and non-real-time recording data for the learner's learning activity,
identifies at least one educational environment and educational content corresponding to the state information, and
transmits a control command for providing at least one of the identified educational environments and educational content to a target device through the communication interface.

2. The electronic device of claim 1, wherein the processor updates prestored state information of the learner based on the acquired real-time sensing value and the non-real-time recording data.

3. The electronic device of claim 1, wherein the processor
identifies a learning state and learning context of the learner based on the acquired real-time sensing value and the non-real-time recording data, and
identifies at least one of an educational environment and educational content optimized for the learner based on the identified learning state and learning context.

4. The electronic device of claim 1, wherein the processor
identifies the biometric data of the learner included in the acquired real-time sensing value,
identifies at least one of a difficulty level and a composition of educational content provided to the learner based on the identified biometric data, and
identifies educational content corresponding to at least one of the identified level of difficulty and the identified composition.

5. The electronic device of claim 4, wherein the processor
identifies a heart rate, stress index, and heart rate variability of the learner based on the identified biometric data,
identifies an arousal level of the learner based on the identified heart rate, stress, and heart rate variability,
identifies the difficulty level corresponding to the arousal level, and
identifies educational content corresponding to the identified level of difficulty.

6. The electronic device of claim 5, wherein
the processor calculates the arousal score corresponding to the arousal level based on Formula 1, and
$$Formula 1 is Arousal score = ( first weight \times heart rate ) + ( second weight \times stress index ) + ( third weight \times 1 / heart rate variability ) ,$$

7. The electronic device of claim 6, wherein the processor,
identifies educational content corresponding to a higher level of difficulty than that of previously provided educational content, or identifies educational content including at least one of entertainment and quizzes when the arousal score is smaller than a first threshold value,
identifies educational content including at least one of supplementary learning and core learning when the arousal score is higher than or equal to the first threshold value and lower than or equal to a second threshold value, and
identifies educational content corresponding to a lower level of difficulty than that of the previously provided educational content, or identifies educational content including at least one of rest and meditation when the arousal score is higher than or equal to the second threshold value.

8. The electronic device of claim 4, wherein
the processor identifies an amount of increase in learning performance of the learner based on Formula 2, and
$$\begin{matrix}Formula 2 is \\ Amount of increase in learning performance = a - b \times distance + noise ,\end{matrix}$$
where a is the amount of increase in learning performance corresponding to a reference biometric data value of the learner, b is a weight corresponding to a degree of learning performance decreasing according to a difference between a measured biometric data value of the learner and the reference biometric data value, the distance is a value of the difference between the measured biometric data value and the reference biometric data value, and the noise is a variable corresponding to randomness.

9. The electronic device of claim 8, wherein
the processor identifies the distance based on Formular 3,
$$\begin{matrix}and Formular 3 is \\ distance = {\sum }_{i = 1}^{N} \vert {f}^{( i )} - {f}_{normal}^{( i )} \vert , and\end{matrix}$$
where f⁽ⁱ⁾ is a measured biometric data value corresponding to an i-th item, and
$${f}_{normal}^{( i )}$$
is a reference biometric data value corresponding to the i-th item.

10. The electronic device of claim 8, wherein the processor
identifies educational content corresponding to a higher level of difficulty than that of the previously provided educational content or identifies educational content including a reward provided to the learner when the distance is smaller than a preset A value,
identifies educational content corresponding to a level of difficulty of the previously provided educational content when the distance is greater than or equal to the A value and smaller than a preset value B,
identifies the educational content corresponding to a level of difficulty that the degree of similarity is greater than or equal to a preset value, compared to the difficulty level of the previously provided educational content when the distance is greater than or equal to the B value and smaller than a preset value C, and
identifies educational content corresponding to a lower level of difficulty than that of the previously provided educational content or identifies educational content including at least one of rest and meditation when the distance is greater than or equal to the C value.

11. A method of operating an electronic device for implementing IoT-based adaptive learning using educational analytics standards, the method comprising:
acquiring a real-time sensing value for a learner's state acquired through at least one sensor from an external device through a communication interface;
acquiring state information of the learner based on the acquired real-time sensing value and non-real-time recording data for the learner's learning activity;
identifying at least one educational environment and educational content corresponding to the state information; and
transmitting a control command for providing at least one of the identified educational environments and educational content to a target device through the communication interface.

12. A non-transitory computer-readable recording medium storing at least one instruction executed by a processor of an electronic device to cause the electronic device to perform the method of claim 11.
